(19) United States
(12) Patent Application Publication
(10) Pub. No.: US 2003/0157042 A1
(43) Pub. Date: Aug. 21, 2003

(54) Eyeshadow comprising a xanthan gum and a mixed silicate

(76) Inventors: Nathalie Collin, Sceaux (FR); Yohann Bichon, Paris (FR); Karl Pays, Chevilly-Larue (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/337,904

(22) Filed: Jan. 8, 2003

Related U.S. Application Data
(60) Provisional application No. 60/348,399, filed on Jan. 16, 2002.

(30) Foreign Application Priority Data
Jan. 9, 2002 (FR) 02 00214

(51) Int. Cl. A61K 7/021; A61K 7/06

(52) U.S. Cl. 424/63; 424/70.6

(57) ABSTRACT

An eyeshadow composition comprising, in a cosmetically acceptable aqueous medium, at least one xanthan gum, at least one mixed silicate, and at least one dyestuff present in an amount of greater than and equal to 10.1% by weight, relative to the total weight of the composition, wherein the eyeshadow composition can have good stability on storage and can produce a homogeneous makeup with staying power.

This application claims benefit of U.S. Provisional Application No. 60/348,399, filed Jan. 16, 2002.

Disclosed herein is an eyeshadow cosmetic composition comprising at least one xanthan gum and at least one mixed silicate. Further disclosed herein is an eyelid makeup process using the composition.

Eyeshadows usually consist of a suitable vehicle and various dyestuffs intended to give a certain colour to the makeup product and after they have been applied to the eyelids. The eyeshadows may be in aqueous or anhydrous form.

The drawback most commonly encountered when using the eyeshadows lies in the fact that, after a certain time, due to the folding of the eyelids, the makeup has a tendency to lose its homogeneity and to become more or less quickly and deeply concentrated in the folds of the eyelids. This can result in an effect that is generally considered unattractive.

Anhydrous eyeshadows, and such as those in the form of compact powder, once applied to the eyelids, have a tendency to wear away over time, especially on account of the mechanical action exerted by the blinking of the eyelids on the makeup deposited. This can result in a loss of material, leading to a deterioration in the makeup and making it necessary for the user to freshen the application of the makeup.

Moreover, for aqueous eyeshadows that have a fluid texture, the dyestuffs may separate out by settling during storage, this instability then making the makeup product unsuitable for homogeneous application to the eyelids.

The present disclosure provides, for example, an eyeshadow with a fluid texture, which can have good stability on storage, for example, for two months at 45° C.

This disclosure also provides, for example, an eyeshadow that can spread easily on the eyelids and form a homogeneous makeup thereon, which has staying power over time.

The inventors have discovered that such an eyeshadow comprises, in an aqueous medium, at least one xanthan gum, at least one mixed silicate, and at least one dyestuff. With these gelling agents, the eyeshadow may be fluid (i.e., it flows under its own weight at a room temperature of 25° C.) and the gel may remain homogeneous over time, for example, on storage for two weeks at 45° C. The gel is compatible with dyestuffs, such as pigments. The eyeshadow thus may have good stability over time.

Moreover, the eyeshadow may spread well on the eyelids and may give a pleasant sensation of freshness, and the makeup result obtained may show homogeneous distribution of the at least one dyestuff. Furthermore, the makeup may have staying power over time with respect to sebum and/or the mechanical action of the movement (blinking) of the eyelids. For example, the at least one dyestuff may not diffuse into the folds of the eyelids and thus may remain distributed on the eyelids over time. The makeup may stay on the eyelids without wearing away.

In addition, the eyeshadow may be easy to remove with water, without the need to use a specific makeup remover.

One embodiment is, for example, an eyeshadow comprising, in a cosmetically acceptable aqueous medium, at least one xanthan gum, at least one mixed silicate, and at least one dyestuff present in an amount greater than or equal to 10.1% by weight, relative to the total weight of the eyeshadow.

Another embodiment is, for example, a cosmetic process for making up eyelids, comprising applying to the eyelids the eyeshadow as defined above.

Another embodiment is, for example, the use of the eyeshadow as defined above to produce a makeup, deposited on the eyelids, which is homogeneous and/or which has staying power.

Another embodiment is, for example, the use of at least one xanthan gum and at least one mixed silicate in an eyeshadow comprising, in a cosmetically acceptable aqueous medium, at least one dyestuff in an amount greater than or equal to 10.1% by weight, relative to the total weight of the eyeshadow, to produce a makeup deposited on eyelids, which is homogeneous and/or which has staying power.

As disclosed herein, the term “mixed silicate” means any silicate chosen from silicates of natural and synthetic origins comprising at least two types of cations chosen from alkali metals (e.g., Na, Li and K), alkaline-earth metals (e.g., Be, Mg and. Ca), transition metals, and aluminium.

The at least one mixed silicate may be of natural or synthetic origin, and may be chosen, for example, from montmorillonites, hectorites, bentonites, beidellite and saponites. In one embodiment, the at least one mixed silicate are chosen, for example, from hectorites, bentonites, and laponites.

To ensure good cosmetic properties, the at least one mixed silicate is, for example, in a finely divided form, such as in the form of particles with a mean particle size ranging from 5 nm to 1000 nm, such as from 20 nm to 600 nm. The at least one mixed silicate particle is generally, for example, in the shape of discs or leaflets. Thus, the term “mean particle size” means herein the mean size in numerical terms of the largest dimension (i.e., length) of these discs or leaflets. Particles in the form of discs or leaflets may have a thickness ranging from about 0.5 to 5 nm.

Phyllosilicates, i.e., silicates with a structure in which the —SiO₄ tetrahedra are organized in leaflets between which are enclosed the metal cations, may, for example, be used.

Laponites may also be used. Laponites are silicates of magnesium, sodium and possibly lithium, having a layered structure similar to that of montmorillonites. Laponite is a synthetic form of the natural mineral known as “hectorite.” The synthetic origin of this family of silicates may have an advantage over the natural form, because it may allow good control of the composition of the product. In addition, laponites may have an advantage of having a particle size that is much smaller than that of natural hectorite and bentonite.

Laponites may include, for example, the products sold by the company Laporte under the names Laponite XLS, Laponite XLG, Laponite RD and Laponite RDS by the company Rockwood (these products are sodium magnesium silicates and sodium lithium magnesium silicates).

The at least one mixed silicate may include, for example, bentonites, such as the product sold under the name Bentone HC by the company Rheox; magnesium aluminium silicates, for example, when hydrated, such as the product sold by the company Vanderbilt Company under the name Veegum Ultra, or calcium silicates and, for example, the product in synthetic form sold by the company Celite under the name Micro-cel C.

The at least one mixed silicate may be present in the composition in an amount, for example, ranging from 0.1% to 10% by weight, further for example, from 0.5% to 5% by weight, and even further for example, from 1% to 4% by weight, relative to the total weight of the composition.

The at least one xanthan gum may be, for example, synthesized by fermentation of sugars with the bacterium xanthomonas campestri and mutants or variants thereof.

The at least one xanthan gum may comprise in their structure three different monosaccharides, namely mannose, glucose and glucuronic acid, in salt form, such as in the form of the sodium salt.

The at least one xanthan gum may have, for example, a viscosity ranging from 0.6 to 1.65 Pa·s (measured at 25° C. using a Brookfield viscometer, type LVT at 60 rpm). It has a molecular weight ranging, for example, from 1 000 000 to 50 000 000.

Such products may include, for example, Keltrol T sold by the company Keltro, Rhodicare S sold by the company Rhodia, and Kelsan S sold by the company Monsanto Chemical.

The at least one xanthan gum may be present, for example, in the composition in an amount ranging from 0.01% to 10% by weight, further for example, from 0.1% to 5% by weight, and even further, for example, from 0.1% to 3% by weight, relative to the total weight of the composition.

The at least one mixed silicate and the at least one xanthan gum may be present in an amount such that the at least one mixed silicate/the at least one xanthan gum weight ratio ranges, for example, from 0.1:1 to 20:1, further for example, from 5:1 to 15:1, and even further, for example, from 8:1 to 13:1.

The eyeshadow composition comprises a cosmetically acceptable aqueous medium, i.e., an aqueous medium that is compatible with the skin of the eyelids. For example, this aqueous medium forms a continuous aqueous phase. The composition may, for example, comprise water in an amount, for example, greater than or equal to 20% by weight, such as from 20% to 89.7% by weight, further such as from 35% to 89.7% by weight, and even further, for example, from 40% to 89.7% by weight, relative to the total weight of the composition.

The aqueous phase may also comprise at least one water-miscible solvent, chosen from, for example, C₂-C₆ monoalcohols, such as ethanol, isopropanol and n-propanol. The at least one water-miscible solvent, such as C₂-C₆ monoalcohols, may be present in an amount, for example, ranging from 0.1% to 20% by weight, such as from 0.5% to 15% by weight, relative to the total weight of the composition.

Since the eyeshadow composition comprises an aqueous phase, it is, for example, in the form of a fluid composition, i.e., in the form of a composition that flows under its own weight, at room temperature (25° C.). The composition may be, for example, in the form of an aqueous gel.

In another embodiment, the composition may also comprise at least one fatty phase dispersed in the aqueous phase, for example, to form an oil-in-water emulsion.

The at least one fatty phase may comprise, for example, at least one oil chosen from, for example, oils of mineral, animal, plant and synthetic origins, hydrocarbon-based oils and silicone oils.

Examples of the hydrocarbon-based oils include, for example, liquid paraffin, liquid petroleum jelly, mink oil, turtle oil, soybean oil, perhydrosqualene, sweet almond oil, beauty-leaf oil, palm oil, grape pip oil, sesame oil, corn oil, groundnut oil, rapeseed oil, sunflower oil, cotton oil, apricot oil, castor oil, avocado oil, jojoba oil, olive oil, and cereal germ oil; lanolic acid, oleic acid, lauric acid, and stearic acid esters; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyidodecyl myristate and 2-octyidodecyl lactate, 2-diethylhexyl succinate, diisostearyl malate, glyceryl triisostearate, and diglyceryl triisostearate; higher fatty acids, such as myristic acid, palmitic acid, stearic acid, behenic acid, oleic acid, linoleic acid, linolenic acid and isostearic acid; higher fatty alcohols, such as cetanol, stearyl alcohol, oleyl alcohol, linoleyl alcohol, linolenyl alcohol, isostearyl alcohol and octyldodecanol.

Examples of the silicone oils include, for example, volatile and non-volatile polymethylsiloxanes (PDMSs) comprising at least one silicone chain chosen from linear and cyclic silicone chains, for example, cyclopolydimethylsiloxanes (cyclomethicones), such as cyclohexasiloxane; polydimethylsiloxanes comprising at least one group chosen from alkyl, alkoxy and phenyl groups, pendent or at the end of the silicone chain, wherein the at least one group comprises from 2 to 24 carbon atoms; phenylsilicones, for example, phenyltrimethicones, phenyldimethicones, phenyltrimethylsiloxyd iphenylsiloxanes, diphenyldimethicones, diphenylmethyldiphenyltrisiloxanes, 2-phenylethyl-trimethylsiloxysilicates and polymethylphenylsiloxanes; and mixtures thereof.

The at least one oil may be present in the composition in an amount, for example, ranging from 0.1% to 30% by weight, further for example, from 1% to 20% by weight, and even further, for example, from 5% to 15% by weight, relative to the total weight of the composition.

The at least one fatty phase may also comprise, for example, at least one other additional fatty substance other than oils, such as waxes, pasty substances and gums.

Examples of the waxes (i.e., fatty substances that are solid at room temperature) include hydrocarbon-based waxes, such as optionally modified beeswax, carnauba wax, candelilla wax, ouricoury wax, Japan wax, cork fibre wax, sugarcane wax, paraffin wax, lignite wax, microcrystalline waxes, lanolin wax, montan wax, ozokerites, polyethylene waxes, the waxes obtained by Fischer-Tropsch synthesis, and C₂₀-C₆₀ fatty alcohols. For example, silicone waxes may also be used, such as alkyls, alkoxys and/or esters of polymethylsiloxane, and mixtures thereof.

The pasty fatty substances include, for example, fatty substances with a melting point ranging from 25 to 45° C. and/or a viscosity at 40° C. ranging from 0.1 to 40 Pa·s, measured using a Contraves TV viscometer equipped with an MS-r3 or MS-r4 spindle rotating at 60 Hz. Examples of the pasty fatty substances include lanolins and lanolin derivatives, for example, acetylated lanolins and oxypropylenated lanolins, with a viscosity at 40° C. ranging from 18 to 21 Pa·s, such as from 19 to 20.5 Pa·s, and/or a melting point from 30 to 55° C., and mixtures thereof. It may be also possible, for example, to use esters of fatty acids or esters of fatty alcohols, such as those comprising from 20 to 65 carbon atoms (melting point of about from 20 to 35° C. and/or viscosity at 40° C. ranging from 0.1 to 40 Pa·s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof. Triglycerides of plant origin which may be used are hydrogenated castor oil derivatives, such as “Thixinr” from Rheox.

Mention may also be made of pasty silicone fatty substances such as polydimethylsiloxanes (PDMSs) comprising pendent chains chosen from those of the alkyl and alkoxy types comprising from 8 to 24 carbon atoms, and having a melting point of 20-55° C., such as stearyldimethicones, for example, those sold by Dow Corning under the trade names DC2503 and DC25514, and mixtures thereof.

The gums that may be used include, for example, silicone gums (e.g., dimethiconol).

The at least one other additional fatty substance may be present, for example, in an amount ranging from 0.1% to 25% by weight, such as from 1% to 15% by weight, relative to the total weight of the composition.

When the eyeshadow composition is in the form of an oil-in-water emulsion, it may comprise at least one emulsifier, which is, for example, appropriately chosen so as to obtain an oil-in-water emulsion. For example, a water-soluble emulsifier may be used, such as a water-soluble emulsifier having an HLB (hydrophilic-lipophilic balance) value of greater than or equal to 10.

The at least one emulsifier may be chosen from, for example, amphoteric, anionic, cationic and nonionic emulsifiers, as well as silicone emulsifiers:

the amphoteric emulsifiers include, for example, N-acylamino acids, such as N-alkylamino acetates and disodium cocoamphodiacetate, and amine oxides, such as stearamine oxide;

the anionic emulsifiers include, for example, acylglutamates, such as disodium hydrogenated tallow glutamate (Amisoft HS-21® sold by the company Ajinomoto); carboxylic acids and salts thereof, such as sodium stearate; phosphoric esters and salts thereof, such as DEA Oleth-10 phosphate; sulphoccinates such as Disodium PEG-5 citrate lauryl sulphosuccinate and Disodium ricinoleamido MEA sulphosuccinate; alkyl ether sulphates, such as sodium lauryl ether sulphate; sulphosuccinates; and isethionates;

the cationic emulsifiers include, for example, alkylimidazolidiniums, such as isostearylethylimidonium ethosulphate; ammonium salts, such as N,N,N-trimethyl-1-docosanaminium chloride (behentrimonium chloride);

the nonionic emulsifiers include, for example, saccharide esters and ethers, such as sucrose stearate, sucrose cocoate, and the mixture of sorbitan stearate and sucrose cocoate sold by the company ICI under the name Arlatone 2121; fatty acid esters (e.g., of a C₈-C₂₄ acid, such as of a C₁₆-C₂₂ acid) of polyols, e.g., of glycerol or of sorbitol, such as glyceryl stearate, poly-2-glyceryl stearate, sorbitan tristearate and glyceryl ricinoleate; glycerol ethers; oxyethylenated and/or oxypropylenated ethers (which may comprise from 1 to 150 groups chosen from oxyethylenated and oxypropylenated groups) of fatty alcohols (e.g., of a C₈-C₂₄ alcohol, such as of a C₁₂-C₁₈ alcohol), such as an oxyethylenated or oxypropylenated ether of lauryl alcohol comprising 25 oxyethylenated groups and 25 oxypropylenated groups (CTFA name: PPG-25 Laureth-25) and an oxyethylenated ether of the mixture of C₁₂-C₁₅ fatty alcohols comprising 7 oxyethylenated groups (CTFA name: C₁₂₋₁₅ Pareth-7); fatty acid esters (e.g., of a C₈-C₂₄ acid, such as of a C₁₆-C₂₂ acid) of polyethylene glycol (which may comprise from 1 to 150 ethylene glycol units), such as PEG-50 stearate and PEG-40 stearate; copolymers of propylene oxide and of ethylene oxide, such as those sold under the names Synperonic PE/F68, Synperonic PE/L44 and Synperonic PE/F127 by the company Uniqema;

the silicone emulsifiers include, for example, dimethicone copolyols, such as those sold under the names DC2-5695 and Q2-5220 by the company Dow Corning, the mixture of cyclomethicone/dimethicone copolyol sold under the name Q2-3225C by the company Dow Corning, and dimethicone copolyol phosphates, such as the product sold under the name Pecosil PS 100 by the company Phoenix Chemical. Silicone emulsifiers that may also be used include, for example, a dimethicone copolyol benzoate, i.e., a partial ester of benzoic acid and of dimethicone copolyol, the latter being a dimethylpolysiloxane polymer comprising side chains chosen from polyoxyethylene and polyoxypropylene side chains. Dimethicone copolyol benzoates that may be used include, for example, those sold under the name Finsolv® SLB-101 and Finsolv® SLB-201 by the company Finetex.

The at least one emulsifier may be present in the composition in an amount, for example, ranging from 0.1% to 10% by weight, further for example, from 0.5% to 8% by weight, and even further, for example, from 0.5% to 5% by weight, relative to the total weight of the composition.

The at least one dyestuff may be chosen, for example, from pulverulent dyestuffs, such as pigments, nacres and flakes, and water-soluble dyestuffs usually used in cosmetic compositions.

The pigments may be chosen from, for example, white and coloured pigments, mineral and organic pigments, and may or may not be interference pigments. Among the mineral pigments that may be mentioned include, for example, titanium dioxide, optionally surface-treated, zirconium oxide and cerium oxide, and also zinc oxide, iron oxide, chromium oxide, manganese violet, ultramarine blue, chromium hydrate and ferric blue. Among the organic pigments that may be mentioned include, for example, carbon black, pigments of D & C type, and lakes based on cochineal carmine or on barium, strontium, calcium or aluminium.

The nacreous pigments may be chosen, for example, from white nacreous pigments, such as mica coated with titanium or with bismuth oxychloride, coloured nacreous pigments, such as titanium mica with iron oxides, titanium mica with, for example, ferric blue or chromium oxide, titanium mica with an organic pigment of the above-mentioned type, and also nacreous pigments based on bismuth oxychloride.

The water-soluble dyestuffs may be chosen, for example, from beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The at least one dyestuff is present, for example, in the composition in an amount greater than or equal to 10.1% by weight relative to the total weight of the composition, such as ranging from 10.1% to 50% by weight, for example, from 10.1% to 40% by weight, and further, for example, from 15% to 30% by weight relative to the total weight of the composition.

The eyeshadow composition may also comprise at least one glycol to allow good wetting of the pigments, i.e., to facilitate their use and their homogeneous dispersion (e.g., absence of aggregates) in the aqueous medium of the composition. The at least one glycol may allow good wetting of the skin, for example, making it easier to spread the composition on the eyelid. As disclosed herein, the term “glycol” means a diol comprising from 2 to 8 carbon atoms, such as from 2 to 4 carbon atoms.

The at least one glycol may be chosen, for example, from propylene glycol, ethylene glycol, 1,3-butylene glycol and dipropylene glycol.

The at least one glycol may be present, for example, in the composition in an amount ranging from 0.1% to 30% by weight, such as from 5% to 20% by weight, relative to the total weight of the composition.

The composition may comprise at least one filler chosen, for example, from those that are well known to persons skilled in the art and that are commonly used in cosmetic compositions. Fillers that may be used include, for example:

talc, which is a hydrated magnesium silicate used in the form of particles generally, for example, less than 40 microns,

micas, which are aluminosilicates of varied composition, which are in the form of flakes from 2 to 200 microns in size, for example, from 5 to 70 microns in size and ranging from 0.1 to 5 microns thick, such as from 0.2 to 3 microns thick, these micas possibly being of natural origin, such as muscovite, margarite, roscoelite, lipidolite or biotite, or of synthetic origin,

starch, for example, rice starch,

kaolin, which is a hydrated aluminium silicate that is in the form of particles of isotropic form generally, for example, less than 30 microns in size,

zinc oxide and titanium oxide, for example, in the form of particles not exceeding a few microns in size,

calcium carbonate, magnesium carbonate and magnesium hydrocarbonate,

microcrystalline cellulose,

silica,

powders of synthetic polymers, such as polyethylene, polyesters (polyethylene isophthalate or terephthalate), polyamides such as those sold under the trade name Nylon or Teflon, and silicone powders.

The composition may comprise at least one cosmetic additive chosen, for example, from preserving agents, fragrances, vitamins, moisturizers, softeners, sunscreens, film-forming polymers, sequestering agents and acidifying and basifying agents.

Needless to say, a person skilled in the art will take care to select this or these optional additional compound(s), and/or the amount thereof, such that the properties of the composition are not, or are not substantially, adversely affected by the envisaged addition.

The composition may be prepared, for example, according to the usual methods of the fields under consideration.

Illustrative, non-exhaustive examples follow.

### EXAMPLE 1

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Propylene glycol | | 7 | g |
| Glycerol | | 5 | g |
| Dimethicone copolyol benzoate | | 3 | g |
| (Finsolv SLB-101 from the company Finetex) | | | |
| Pigments | | 10 | g |
| Bismuth oxychloride | | 5 | g |
| Preserving agents | qs | | |
| Water | qs | 100 | g |

This fluid eyeshadow is stable on storage for 2 months at 45° C. It spreads easily on the eyelids, giving a sensation of freshness, and forms a homogeneous makeup that does not form streaks and that has staying power, for example, good resistance to sebum and to the mechanical action (blinking) of the eyelids. It is easy to remove with water.

### EXAMPLE 2

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Propylene glycol | | 7 | g |
| Glycerol | | 5 | g |
| Phenylsilicone | | 10 | g |
| (Belsil PDM 1000 from the company Wacker) | | | |
| Dimethicone copolyol benzoate | | 3 | g |
| (Finsolv(Finsolv SLB-101 from the company Finetex) | | | |
| Pigments | | 10 | g |
| Bismuth oxychloride | | 5 | g |
| Preserving agents | qs | | |
| Water | qs | 100 | g |

This fluid eyeshadow is stable on storage for 2 months at 45° C. It spreads easily on the eyelids, giving a sensation of freshness, and forms a homogeneous makeup with good staying power.

### EXAMPLE 3

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Glycerol | | 5 | g |
| Propylene glycol | | 7 | g |
| Mixture of polydimethylsiloxane containing | | 10 | g |
| αω-hydroxyl groups and cyclopentadimethyl- | | | |
| siloxane groups (15/85) | | | |
| (DC2-9071 from Dow Corning) | | | |
| Dimethicone copolyol benzoate | | 3 | g |
| (Finsolv SLB-101 from the company Finetex) | | | |
| Pigments | | 15 | g |
| Bismuth oxychloride | | 5 | g |
| Preserving agents | qs | | |
| Water | qs | 100 | g |

This eyeshadow forms a homogeneous makeup on the eyelids and withstands the movements of the eyelids.

### EXAMPLE 4

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Propylene glycol | | 7 | g |
| Glycerol | | 5 | g |
| Phenylsilicone | | 10 | g |
| (Belsil PDM 1000 from the company Wacker) | | | |
| Dimethicone copolyol | | 3 | g |
| (Q2-5220 from Dow Corning) | | | |
| Pigments | | 15 | g |
| Bismuth oxychloride | | 5 | g |
| Preserving agents | qs | | |
| Water | qs | 100 | g |

This eyeshadow is easy to apply to the eyelids and forms a homogeneous makeup with good staying power.

### EXAMPLE 5

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Propylene glycol | | 7 | g |
| Glycerol | | 5 | g |
| Isononyl isononate | | 10 | g |
| Dimethicone copolyol benzoate | | 3 | g |
| (Finsolv SLB-101 from the company Finetex) | | | |
| Pigments | | 15 | g |
| Bismuth oxychloride | | 5 | g |
| Preserving agents | qs | | |
| Water | qs | 100 | g |

This eyeshadow is easy to apply to the eyelids and forms a homogeneous makeup with good staying power.

### EXAMPLE 6

An eyeshadow having the composition below was prepared:

| | | | |
|---|---|---|---|
| Xanthan gum | | 0.3 | g |
| Sodium magnesium silicate | | 3 | g |
| (Laponite XLG from the company Rockwood) | | | |
| Ethanol | | 10 | g |
| Propylene glycol | | 7 | g |
| Glycerol | | 5 | g |
| Isononyl isononate | | 10 | g |
| Polyethylene glycol stearate (40 EO) | | 5 | g |
| Pigments | | 15 | g |
| Bismuth oxychloride | | 5 | g |
| Preserving agents | qs | | |
| Water | qs | 100 | g |

This eyeshadow is easy to apply the eyelids and forms a homogeneous makeup with good staying power.

What is claimed is:

1. An eyeshadow composition comprising, in a cosmetically acceptable aqueous medium, (a) at least one xanthan gum, (b) at least one mixed silicate, and (c) at least one dyestuff, wherein the at least one dyestuff is present in an amount greater than or equal to 10.1% by weight, relative to the total weight of the composition.

2. The eyeshadow composition according to claim 1, wherein the at least one mixed silicate is in a finely divided form with a mean particle size ranging from 15 nm to 1000 nm.

3. The eyeshadow composition according to claim 1, wherein the at least one mixed silicate is chosen from phyllosilicates.

4. The eyeshadow composition according to claim 1, wherein the at least one mixed silicate is chosen from hectorites.

5. The eyeshadow composition according to claim 1, wherein the at least one mixed silicate is chosen from laponites.

6. The eyeshadow composition according to claim 1, wherein the at least one mixed silicate is present in an amount ranging from 0.1% to 10% by weight, relative to the total weight of the composition.

7. The eyeshadow composition according to claim 6, wherein the at least one mixed silicate is present in an amount ranging from 0.5% to 5% by weight, relative to the total weight of the composition.

8. The eyeshadow composition according to claim 7, wherein the at least one mixed silicate is present in an amount ranging from 1% to 4% by weight, relative to the total weight of the composition.

9. The eyeshadow composition according to claim 1, wherein the at least one xanthan gum is present in an amount ranging from 0.01% to 10% by weight, relative to the total weight of the composition.

10. The eyeshadow composition according to claim 9, wherein the at least one xanthan gum is present in an amount ranging from 0.1% to 5% by weight, relative to the total weight of the composition.

11. The eyeshadow composition according to claim 10, wherein the at least one xanthan gum is present in an amount ranging from 0.1% to 3% by weight, relative to the total weight of the composition.

12. The eyeshadow composition according to claim 1, wherein a weight ratio of the at least one mixed silicate to the at least one xanthan gum ranges from 0.1:1 to 20:1.

13. The eyeshadow composition according to claim 12, wherein the weight ratio of the at least one mixed silicate to the at least one xanthan gum ranges from 5:1 to 15:1.

14. The eyeshadow composition according to claim 13, wherein the weight ratio of the at least one mixed silicate to the at least one xanthan gum ranges from 8:1 to 13:1.

15. The eyeshadow composition according to claim 1, further comprising at least 20% by weight of water, relative to the total weight of the composition.

16. The eyeshadow composition according to claim 15, wherein the water is present in an amount ranging from 20% to 89.7% by weight, relative to the total weight of the composition.

17. The eyeshadow composition according to claim 16, wherein the water is present in an amount ranging from 35% to 89.7% by weight, relative to the total weight of the composition.

18. The eyeshadow composition according to claim 17, wherein the water is present in an amount ranging from 40% to 89.7% by weight, relative to the total weight of the composition.

19. The eyeshadow composition according to claim 1, further comprising at least one water-miscible solvent chosen from C₂-C₆ monoalcohols.

20. The eyeshadow composition according to claim 19, wherein the at least one water-miscible solvent is chosen from ethanol, isopropanol, and n-propanol.

21. The eyeshadow composition according to claim 19, wherein the at least one water-miscible solvent is present in an amount ranging from 0.1% to 20% by weight, relative to the total weight of the composition.

22. The eyeshadow composition according to claim 21, wherein the at least one water-miscible solvent is present in an amount ranging from 0.5% to 15% by weight, relative to the total weight of the composition.

23. The eyeshadow composition according to claim 1, wherein the at least one dyestuff is chosen from pigments, nacres, flakes, and water-soluble dyestuffs.

24. The eyeshadow composition according to claim 23, wherein the at least one dyestuff is chosen from titanium dioxide, zirconium oxide, cerium oxide, zinc oxide, iron oxide, chromium oxide, manganese violet, ultramarine blue, chromium hydrate, ferric blue, carbon black, lakes based on cochineal carmine, lakes based on barium, strontium, calcium and aluminium; mica coated with titanium, mica coated with bismuth oxychloride, titanium mica with iron oxides, titanium mica with ferric blue, titanium mica with chromium oxide, and nacreous pigments based on bismuth oxychloride.

25. The eyeshadow composition according to claim 1, wherein the at least one dyestuff is present in an amount ranging from 10.1% to 50% by weight, relative to the total weight of the composition.

26. The eyeshadow composition according to claim 25, wherein the at least one dyestuff is present in an amount ranging from 10.1% to 40% by weight, relative to the total weight of the composition.

27. The eyeshadow composition according to claim 26, wherein the at least one dyestuff is present in an amount ranging from 15% to 30% by weight, relative to the total weight of the composition.

28. The eyeshadow composition according to claim 1, further comprising at least one glycol comprising from 2 to 8 carbon atoms

29. The eyeshadow composition according to claim 28, wherein the at least one glycol is chosen from propylene glycol, ethylene glycol, 1,3-butylene glycol and dipropylene glycol.

30. The eyeshadow composition according to claim 28, wherein the at least one glycol is present in an amount ranging from 0.1% to 30% by weight, relative to the total weight of the composition.

31. The eyeshadow composition according to claim 30, wherein the at least one glycol is present in an amount ranging from 5% to 20% by weight, relative to the total weight of the composition.

32. The eyeshadow composition according to claim 1, wherein the composition is in the form of an oil-in-water emulsion.

33. The eyeshadow composition according to claim 32, comprising a fatty phase comprising at least one oil.

34. The eyeshadow composition according to claim 33, wherein the at least one oil is chosen from hydrocarbon-based oils and silicone oils.

35. The eyeshadow composition according to claim 33, wherein the at least one oil is present in an amount ranging from 0.1% to 30% by weight, relative to the total weight of the composition.

36. The eyeshadow composition according to claim 35, wherein the at least one oil is present in an amount ranging from 1% to 20% by weight, relative to the total weight of the composition.

37. The eyeshadow composition according to claim 36, wherein the at least one oil is present in an amount ranging from 5% to 15% by weight, relative to the total weight of the composition.

38. The eyeshadow composition according to claim 33, wherein the fatty phase comprises at least one additional fatty substance chosen from waxes, gums and pasty substances.

39. The eyeshadow composition according claim 38, wherein the at least one additional fatty substance is present in an amount ranging from 0.1% to 25% by weight, relative to the total weight of the composition.

40. The eyeshadow composition according claim 39, wherein the at least one additional fatty substance is present in an amount ranging from 1% to 15% by weight, relative to the total weight of the composition.

41. The eyeshadow composition according to claim 33, further comprising at least one emulsifier.

42. The eyeshadow composition according to claim 41, wherein the at least one emulsifier is chosen from N-acylamino acids, amine oxides, acylglutamates, carboxylic acids and salts thereof, phosphoric esters and salts thereof, sulphoccinates, alkyl ether sulphates, sulphosuccinates, isethionates, alkylimidazolidiniums, ammonium salts, saccharide esters and ethers, fatty acid esters of polyols, glycerol ethers, oxyethylenated and oxypropylenated ethers of fatty alcohols, fatty acid esters of polyethylene glycol, copolymers of propylene oxide and of ethylene oxide, dimethicone copolyols, dimethicone copolyol phosphates and dimethicone copolyol benzoates.

43. The eyeshadow composition according to claim 41, wherein the at least one emulsifier is present in an amount ranging from 0.1% to 10% by weight, relative to the total weight of the composition.

44. The eyeshadow composition according to claim 43, wherein the at least one emulsifier is present in an amount ranging from 0.5% to 8% by weight, relative to the total weight of the composition.

45. The eyeshadow composition according to claim 44, wherein the at least one emulsifier is present in an amount ranging from 0.5% to 5% by weight, relative to the total weight of the composition.

46. The eyeshadow composition according to claim 1, further comprising at least one cosmetic additive chosen from fillers, preserving agents, fragrances, vitamins, moisturizers, softeners, sunscreens, film-forming polymers, sequestering agents, and acidifying and basifying agents.

47. A process for making up eyelids, comprising applying to the eyelids an eyeshadow composition comprising, in a cosmetically acceptable aqueous medium:
(a) at least one xanthan gum;
(b) at least one mixed silicate; and
(c) at least one dyestuff, wherein the at least one dyestuff is present in an amount greater than or equal to 10.1% by weight, relative to the total weight of the composition.

48. A makeup deposited on eyelids comprising at least one eyeshadow composition;
wherein the at least one eyeshadow composition comprises, in a cosmetically acceptable aqueous medium:
(a) at least one xanthan gum;
(b) at least one mixed silicate; and
(c) at least one dyestuff, wherein the at least one dyestuff is present in an amount greater than or equal to 10.1% by weight, relative to the total weight of the composition;
wherein the makeup deposited on the eyelids has at least one characteristic chosen from characteristics of being homogeneous and having staying power.

49. A method for depositing a makeup on eyelids comprising applying to the eyelids an eyeshadow composition;
wherein the eyeshadow composition comprises, in a cosmetically acceptable aqueous medium:
(a) at least one xanthan gum;
(b) at least one mixed silicate; and
(c) at least one dyestuff, wherein the at least one dyestuff is present in an amount greater than or equal to 10.1% by weight, relative to the total weight of the composition;
wherein the makeup deposited on the eyelids has at least one characteristic chosen from characteristics of being homogeneous and having staying power.

50. A method for producing a makeup deposited on eyelids comprising including in an eyeshadow composition (a) at least one xanthan gum; and (b) at least one mixed silicate;
wherein the eyeshadow composition comprises, in a cosmetically acceptable aqueous medium, at least one dyestuff present in an amount greater than or equal to 10.1% by weight, relative to the total weight of the composition; and applying to the eyelids the eyeshadow composition,
wherein the make up has at least one characteristic chosen from characteristics of being homogeneous and having staying power.

51. An eyeshadow composition for producing a makeup deposited on eyelids, comprising, in a cosmetically acceptable aqueous medium, (a) at least one xanthan gum, (b) at least one mixed silicate, and (c) at least one dyestuff, wherein the at least one dyestuff is present in an amount greater than or equal to 10.1% by weight, relative to the total weight of the composition;
wherein the eyeshadow composition is effective in producing the makeup, which has at least one characteristic chosen from characteristics of being homogeneous and having staying power.
